(19) United States
(12) Patent Application Publication
(10) Pub. No.: US 2006/0160224 A1
(43) Pub. Date: Jul. 20, 2006

(54) Fish labeling system

(76) Inventors: Michelle Diane Benoit, Santa Cruz, CA (US); Teresa Lin Ish, Emeryville, CA (US)

Correspondence Address: Nathaniel Gordon-Clark, 1025 North Calvert Street, Baltimore, MD, 21202, US

(21) Appl. No.: 11/331,957

(22) Filed: Jan. 13, 2006

Related U.S. Application Data
(60) Provisional application No. 60/644,196, filed on Jan. 14, 2005.

(51) Int. Cl. G01N 33/00

(52) U.S. Cl. 436/2

(57) ABSTRACT

The invention is a system to allow a fishmonger or other fish seller to label fish for the sustainable use of that fish. The provider of the invention collects data and color-codes the data upon a flow chart. The flow chart combines the data into a single color code. The fishmonger can then read the flow chart and label the fish without needed to review the data for him or her self. Alternative versions could be developed that produce and distribute the flow chart on a computer or use other products rather than fish and other data rather than sustainability.

### CROSS-REFERENCE TO RELATED APPLICATIONS

This application claims the benefit of Provisional Patent Application Ser. No. 60/644,196, filed Jan. 14, 2005 by the present inventors.

### FEDERALLY SPONSORED RESEARCH

not applicable

### SEQUENCE LISTING OR PROGRAM

not applicable

### FIELD OF INVENTION

This invention relates to the labeling of products. More specifically it relates to the labeling of fish and seafood. Even more specifically it relates to the labeling of fish and seafood for the environmentally sustainable harvesting and farming of the fish or seafood.

### BACKGROUND OF THE INVENTION

This invention is a system to inform the public about the sustainable harvesting and farming of fish and seafood resources. Previous efforts to inform the public on sustainable harvesting and farming of fish have focused on directly informing each person about the subject. The usual method is to supply individual consumers with cards and pamphlets that inform the reader about what fish are sustainably harvested or farmed. This has required the consumer to either remember this information or to carry the paperwork with them to the fish market. In practice, consumers do not remember this information and often forget to even carry the written materials with them.

In addition, the sustainability of each fish changes constantly. Thus information told to consumers can be outdated within a year or even a month. Since the consumer does not receive regular updates, the consumer's information will become outdated and the consumer will no longer be able to make sustainable choices when purchasing fish. Furthermore, consumers are aware of this problem and thus further discouraged from remembering sustainability information or carry such information on printed materials.

Previous inventions have primarily been new and adaptable forms of labels. Examples are Gribb, U.S. Pat. No. 4,580,360, and Banning, U.S. Pat. No. 6,226,909. Both describe new kinds of labels, but not s system for collecting data and displaying the combined result of the data. The present invention could be used in conjunction with either Gribb's or Banning's labels, or with any other form of labels. Kiefer, U.S. Pat. No. 5,558,742, describes a system for combining nutritional information on a label. The Kiefer labeling system collects nutritional information and then puts a symbol on the label for each nutrient. The relative size of the nutrient symbol indicates its relative amount. The Kiefer system does not produce a single symbol or use a color-coded system. Furthermore the Kiefer system is for nutritional information only.

### SUMMARY OF INVENTION

This invention is an adaptable fish labeling system and its method of use. The labeling system is a way to convey complex information to consumers without requiring any study by the consumer. The labeling system involves the collection of data by the provider of the labeling system. The provider then reduces the data to a flow chart. This flow chart will produce a coded label for any given fish from any given source. A fishmonger can read the chart using basic information about the actual fish that the fishmonger is selling and label each fish with a coded label. The coded label then conveys to consumers the proper conclusion that would have been derived from a full study of the original complex data.

For the purpose of this application, fish also refers to all seafood and aquatic plants and aquatic invertebrates. Thus fish includes, but not limited to, crabs, lobster, clams, muscles, oysters, sea cucumbers, abalone, and seaweed. Also for the purpose of this application, a fishmonger is any seller of fish or seafood. A fishmonger can be a retailer or a wholesaler, and can operate from a permanent or temporary site. For the purpose of this application, use of fish means the harvesting, farming and selling of fish and seafood.

### PREFERRED EMBODIMENT

The preferred embodiment of this labeling system is a means of making consumers aware of the environmental impacts of fish consumption and of allowing consumers to choose to eat the fish that is associated with the least adverse environmental impact. The preferred embodiment is a color-coded system.

The flow chart is created by gathering data from a source that has detailed information on the environmental sustainability of different fishing methods and of removing different fish from the environment. In some cases, this information is divided into area of origin of the fish as well. As an example, but not meant as a limitation of any kind, the Monterey Bay Aquarium's Seafood Watch Program could be a source of data. The data could be gathered from multiple sources.

This data is reduced to a red, yellow and green color code. The red code is for information that indicates unsustainability. The green code is for information that indicates sustainability. The yellow code is for information that indicates a partially sustainable use, either because the fishery is recovering, one of the ranking criteria is unsustainable, or for cases where the information is missing. Alternatively, the color black could be used for cases of extreme unsustainability. This color-coded information is then placed in a hierarchy. The hierarchy balances information such as the type of fish, the origin of the fish, method of catch, discharge from aquaculture, disease risk from aquaculture, chance of escape from a fish farm and source of food for the fish farm.

These colors are then averaged by use of the flow chart. The flow chart starts with a fish, evaluates its origins, role in the ecosystem, and each relevant factor in removing the fish from the ecosystem and bringing it to market. In the case of wild caught fish these factors are type of fish and method of catch. In the case of farm raised fish the factors are discharge from aquaculture, disease risk from aquaculture, chance of escape from a fish farm and source of food for the fish farm. Each factor has a color. Following the flow chart creates a single color that is then used to label the fish and inform the consumer about the sustainability and thus desirability of how this fish was brought to market.

Its provider assembles the flow chart. The flow chart is then distributed to fishmongers. These fishmongers are educated on how to use the flow chart, but the flow chart is simple enough to be used without instruction. The fishmonger takes fish that is for sale and labels it. The fishmonger labels it by finding the type, origin, and catch method of the fish on the flow chart and then going through the flow chart. At each branching the fishmonger decides which way to turn based upon the knowledge that the fishmonger has about the fish for sale. With each decision the color is averaged and produces a final color that the fishmonger uses to label the fish.

### OTHER EMBODIMENTS

Other versions of the invention involve the use of a computer system to determine the proper label color for the fishmonger. This computer-based variation could use a single program to determine the proper color-coded label for a given fish. The fishmonger could enter the required data and receive a color code determination. Alternatively, the program could contain data about national or regional averages for certain factors that the fishmonger may not know. As an example, the program could contain the national average for all aquiculture operations regarding the five relevant factors for the labeling system. Alternatively, the program could contain specific data about certain producers of farmed fish. Thus when the fishmonger did not know the answer to certain factors in the chart, the program would contain data that could fill in the gaps in the fishmongers knowledge about the fish, its environmental factors and factors in the farming of the fish. Other versions could use an internet-based program that the fishmonger could log onto and both acquire proper color codes for certain fish and acquire data on factors that the fishmonger does not know about a certain fish, its environmental factors and the factors in the farming of that fish.

The labeling system could also include charts that contain data about the sources of the fish that the fishmonger is trying to label. The charts could contain data about national or regional averages for certain factors that the fishmonger may not know. As an example, the charts could contain the national average for all aquiculture operations regarding the five relevant factors for the labeling system. Alternatively, the charts could contain specific data about certain producers of farmed fish. Thus when the fishmonger did not know the answer to certain factors in the labeling chart, the additional charts would contain data that could fill in the gaps in the fishmongers knowledge about the fish, its environmental factors and factors in the farming of the fish.

Other versions of the invention could be used to label other products for environmental information. Examples, meant to illustrate and not as a limitation in any way, would be meat and produce. Persons skilled in the art will recognize other possibilities. The invention could also be used to label fish or other products for non-environmental information. Examples mean to illustrative and not as a limitation, might be safety information or labor union involvement information. Persons skilled in the art will recognize other possibilities.

### BRIEF DESCRIPTION OF THE DRAWINGS

The accompanying drawings, which are incorporated in and form part of the specification, illustrate the embodiments of the present invention and, together with the description, serve to explain the principles of the invention.

FIG. 1 is a picture of a flow chart that could be distributed to fishmongers.

FIG. 2 is a picture of the averaging chart that averages different color code combinations.

FIG. 3 is a picture of a flow chart that could be distributed to fishmongers.

### DETAILED DESCRIPTION OF THE DRAWINGS

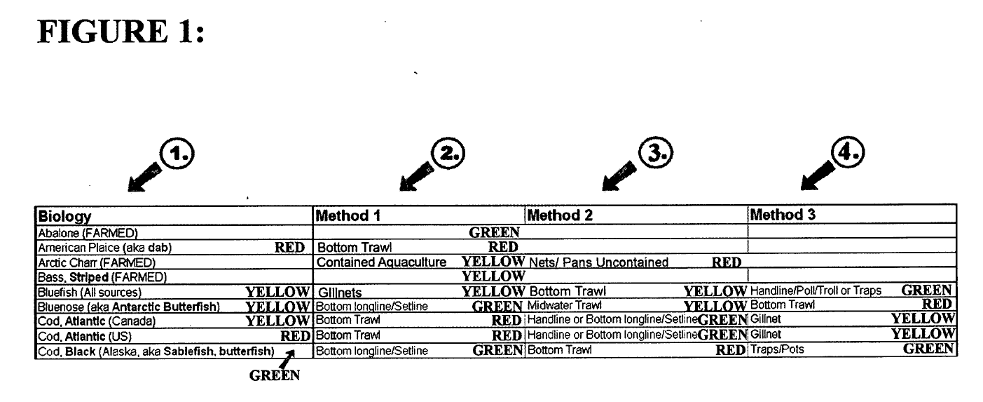

FIG. 1 illustrates a possible flow chart for fishmongers. The chart consists of four columns. The first column is Biology 1 that states the biology of fish. This is the type of fish and in some cases the location of origin. The remaining three columns are the methods of catch or the method of farming. These columns are method 1 2, method 2 3 and method 3 4. Each entry in the biology column 1 and the method columns 2 3 4 is color-coded. In this figure, the color is written in each entry in bold. The invention envisions that each entry will be printed in its color instead of stating the color or in addition to stating the color. Note that some fish will have only one or two catch or farming methods and the remaining method columns are blank. Also note that this figure uses a smaller version of the flow chart that contains only nine rows. The invention envisions using larger flow charts with more rows including charts large enough to include all fish sold commercially or all fish in existence.

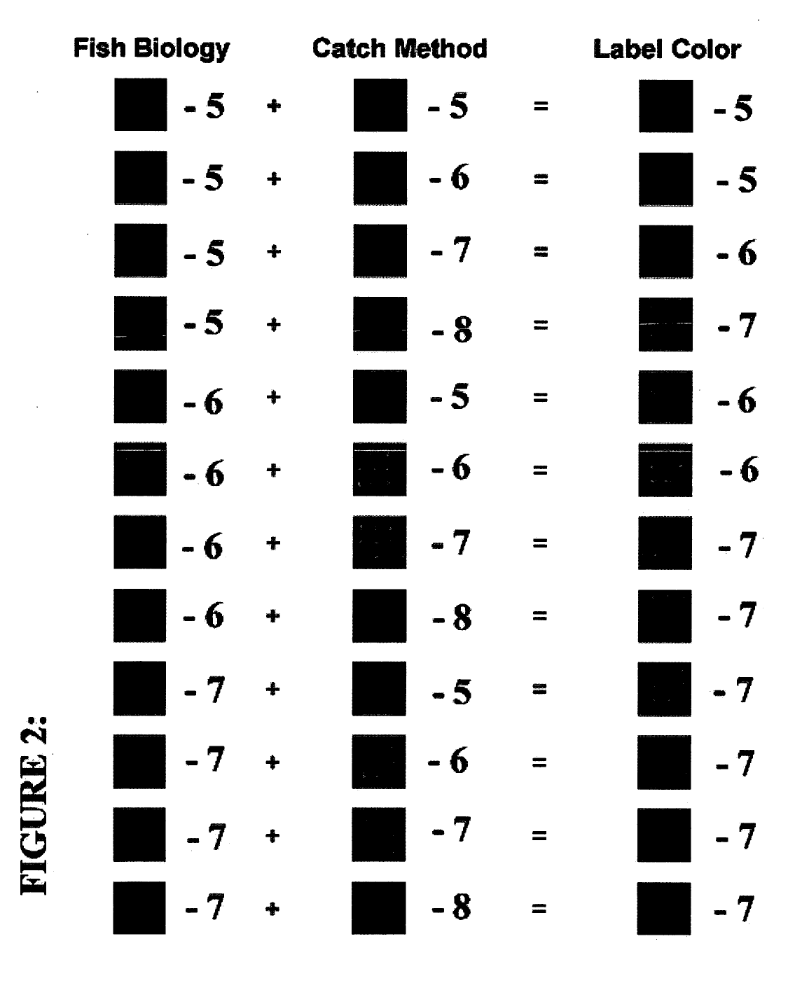

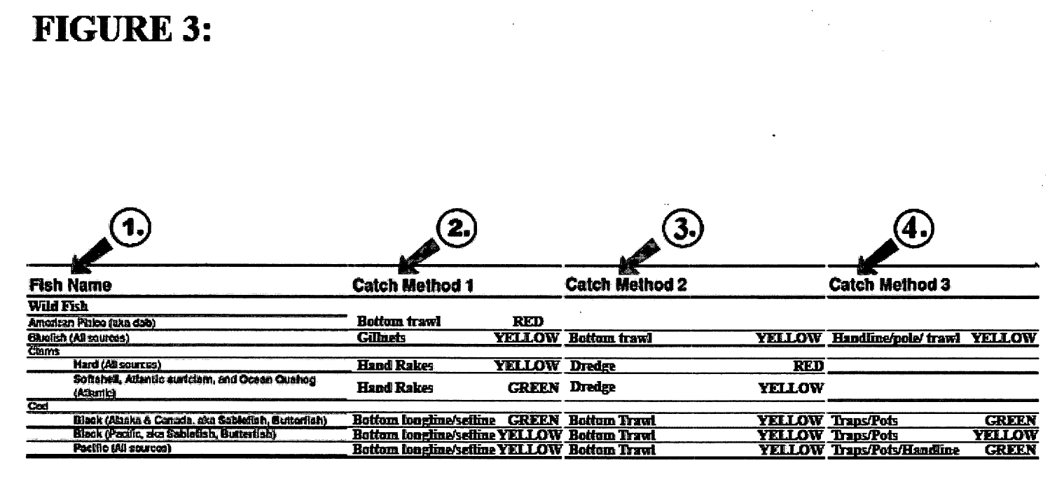

FIG. 2 illustrates the averaging method for averaging color codes. The color codes are green 5, yellow 6, red 7 and black 8. The figure shows the outcome of averaging any two colors. For instance, averaging a green 5 with a green 5 results in a green 5. Another example, averaging a green 5 with a yellow 6 results in a green 5. On the other hand, averaging a green 5 with a red 7 results in a yellow 6. Another example, averaging a yellow 6 with a red 7 results in a red 7. As a final example, when a black 8 is combined with any color results in a red 7. In the case of a fishmonger who is using the flow chart shown in FIG. 1, the fishmonger could use this averaging chart to average the combination of a biology 1 color code with a method 1 2 color code and thus know how to label the fish for sustainability. In the case of a fishmonger who is using a flow chart prepared in the manner of FIG. 3, this averaging chart shows the method used to determine the color code on the FIG. 3 flow chart.

FIG. 3 illustrates a possible flow chart for fishmongers. The chart consists of four columns. The first column is Biology 1 that states the biology of fish. This is the type of fish and in some cases the location of origin. The remaining three columns are the methods of catch. These columns are method 1 2, method 2 3 and method 3 4. In this version of the flow chart, only the method of catch is color-coded. In this version, a fishmonger does not need to do the final averaging step, but looks at the biology 1 and then looks across the chart at the method to determine the color code. In this figure, the color is written in each entry in bold. The invention envisions that each entry will be printed in its color instead of stating the color or in addition to stating the color. As an example, a fishmonger with bluefish that was caught using gillnets will look at the bluefish entry in biology 1 and then move to method 1 2 which is gillnets. This entry has a color code of yellow. The fishmonger will know to label these gillnet caught bluefish as yellow for sustainability. Note that some fish will have only one or two catch or farming methods and the remaining method columns are blank. Also note that this figure uses a smaller version of the flow chart that contains only nine rows. The invention envisions using larger flow charts with more rows including charts large enough to include all fish sold commercially or all fish in existence.

Although this invention has been illustrated by reference to specific embodiments, it will be apparent to those skilled in the art that various changes and modification may be made which clearly fall within the scope of the invention. The invention is intended to be protected broadly within the spirit and scope of the appended claims.

What is claimed is:

1. A labeling system comprising:
a product,
a chart,
data collected from a source,
a color-coding system that denotes various levels of sustainable use,
so that the color-coding system is based upon the collected data and the flow chart averages the levels of sustainable use by averaging the coded colors to produce a single color that can be placed as a label on the product.

2. The labeling system of claim 1 where the product is seafood.

3. The labeling system of claim 1 where the product is fish

4. The labeling system of claim 1 where the product is selected from list that includes fish, seafood, shellfish, claims, shrimp, prawns, oysters, crayfish, crabs, eel, lobster, sea cucumber, abalone, marine fish, freshwater fish, aquatic plants and aquatic invertebrates.

5. The labeling system of claim 1 where the product is meat.

6. The labeling system of claim 1 where the product is vegetables.

7. The labeling system of claim 1 where the chart is a flow chart.

8. The labeling system of claim 1 where the data are data on the environmentally sustainable use of the product.

9. The labeling system of claim 1 where the data are from more than one source.

10. The labeling system of claim 1 where the color coding system uses green to indicate a sustainable use, yellow to indicate a partially sustainable use, and red to indicate an unsustainable use.

11. The labeling system of claim 1 where the chart is produced using a computer.

12. The labeling system of claim 1 where the chart is distributed using a computer.

13. The labeling system of claim 12 where the chart is distributed to consumers.

14. The labeling system of claim 1 where

15. A labeling system comprising:
a flow chart;
fish;
data about the environmentally sustainable farming and catching of fish;
a system of codes in the color green, yellow and red, where green indicates a sustainable use of fish, yellow indicates a partially sustainable use of fish and red indicates an unsustainable use of fish;
a system to assign each piece of data a color code and place each piece of data on the flow chart so that a single color code can be determined from multiple pieces of color-coded data;
and a system of combining color codes so that green and yellow combine to equal green, green and red combine to equal yellow, and yellow and red combine to equal red and two codes of the same color combine to equal that color;
so that a fishmonger can use the flow chart to determine the sustainability of the use of a given fish and label that fish with a single color-coded label to inform the consumer about the sustainable use of that fish.
